(19) United States
(12) Patent Application Publication
(10) Pub. No.: US 2026/0031203 A1
(43) Pub. Date: Jan. 29, 2026

(54) LARGE LANGUAGE MODEL BASED HEALTH MONITORING

(71) Applicant: Samsung Electronics Co., Ltd., Suwon-si (KR)

(72) Inventors: Han Wang, Allen, TX (US); Hao Chen, Allen, TX (US); Lianjun Li, McKinney, TX (US)

(21) Appl. No.: 19/226,045

(22) Filed: Jun. 2, 2025

Related U.S. Application Data
(60) Provisional application No. 63/674,730, filed on Jul. 23, 2024.

(51) Int. Cl. G16H 10/60; G06F 40/30; G06Q 10/0631

(52) U.S. Cl. CPC G16H 10/60; G06F 40/30; G06Q 10/06316

(57) ABSTRACT

A method includes obtaining, by an electronic device, one or more user data types from one or more health devices communicatively coupled to the electronic device, including, by the electronic device, the one or more user data types in a user database, receiving, by the electronic device, an input via a user interface, and generating, using large language model (LLM) agents associated with the user database, user-specific health information based on the input, one or more user data types and a user-specific healthcare data, and providing, by the electronic device, the user-specific health information via the user interface. The user-specific healthcare data may be accessed from a healthcare database communicatively coupled to one of the LLM agents.

### CROSS-REFERENCE TO RELATED APPLICATION(S) AND CLAIM OF PRIORITY

This application claims priority under 35 U.S.C. § 119(e) to U.S. Provisional Patent Application No. 63/674,730 filed on Jul. 23, 2024. The above-identified provisional patent application is hereby incorporated by reference in its entirety.

### TECHNICAL FIELD

This disclosure relates generally to health monitoring systems. More specifically, this disclosure relates to a large language model based health monitoring system and method.

### BACKGROUND

Recently, the healthcare monitoring systems market has experienced a significant growth propelled by technological advancements, increasing health care demands and costs, demographic shifts and a growing focus on preventive cares. For example, chronic conditions such as diabetes, cardiovascular diseases, respiratory disorders, or sleep disorders are increasing globally, necessitating continuous monitoring. The global geriatric population is expanding, increasing demand for monitoring devices, especially home-based healthcare. The technological innovations such as artificial intelligence (AI), machine learning (ML), Internet of Things (IoT) and wearable devices are enhancing the health monitoring systems, allowing real-time diagnostics, predictive analytics and remote monitoring. For example, the advent of wearable devices such as smartwatches, fitness trackers or health monitors has revolutionized the way people track their health and offer continuous vital sign and health state tracking. The COVID-19 pandemic has accelerated the adoption of remote patient monitoring (RPM) systems, which enable healthcare providers to remotely monitor patients with chronic conditions and reduce hospital visits. The growing aging population and rising healthcare costs have led to an increased demand for home healthcare services, driving the increase in the adoption of health monitoring systems. In 2020, the global health monitoring systems market was valued at $34.4 billion and is expected to reach $93.4 billion by 2027, growing at a CAGR (Compound Annual Growth Rate) of 15.4% during the forecast period. The RPM market is expected to reach $1.4 billion by 2027, growing faster at a CAGR of 20.6% from 2020 to 2027. Such growth trajectory of the health monitoring systems market indicates their critical role in modern healthcare, enhancing patient outcomes and reducing the healthcare costs.

### SUMMARY

This disclosure provides a large language model based health monitoring system and method.

In one embodiment, a method is provided. The method includes obtaining, by an electronic device, one or more user data types from one or more health devices communicatively coupled to the electronic device, including, by the electronic device, the one or more user data types in a user database, receiving, by the electronic device, an input via a user interface, and generating, using large language model (LLM) agents associated with the user database, user-specific health information based on the input, one or more user data types and a user-specific healthcare data, and providing, by the electronic device, the user-specific health information via the user interface. The user-specific healthcare data may be accessed from a healthcare database communicatively coupled to one of the LLM agents.

In another embodiment, an electronic device includes a memory and a processor operably coupled to the memory. The processor is configured to obtain one or more user data types from one or more health devices communicatively coupled to the electronic device, include the one or more user data types in a user database, receive an input via a user interface, generate, using large language model (LLM) agents associated with the user database, user-specific health information based on the input, the one or more user data types and a user-specific healthcare data, and provide the user-specific health information via the user interface. The user-specific healthcare data may be accessed from a healthcare database communicatively coupled to one of the LLM agents.

In yet another embodiment, a non-transitory computer readable medium embodying a computer program is provided. The computer program includes program code that, when executed by a processor of an electronic device, causes the electronic device to: obtain one or more user data types from one or more health devices communicatively coupled to the electronic device, include the one or more user data types in a user database, receive an input via a user interface, generate, using large language model (LLM) agents associated with the user database, user-specific health information based on the input, the one or more user data types and a user-specific healthcare data, and provide the user-specific health information via the user interface. The user-specific healthcare data accessed from a healthcare database may be communicatively coupled to one of the LLM agents.

Other technical features may be readily apparent to one skilled in the art from the following figures, descriptions, and claims.

Before undertaking the DETAILED DESCRIPTION below, it may be advantageous to set forth definitions of certain words and phrases used throughout this patent document. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

As used here, terms and phrases such as “have,” “may have,” “include,” or “may include” a feature (like a number, function, operation, or component such as a part) indicate the existence of the feature and do not exclude the existence of other features. Also, as used here, the phrases “A or B,” “at least one of A and/or B,” or “one or more of A and/or B” may include all possible combinations of A and B. For example, “A or B,” “at least one of A and B,” and “at least one of A or B” may indicate all of (1) including at least one A, (2) including at least one B, or (3) including at least one A and at least one B. Further, as used here, the terms “first” and “second” may modify various components regardless of importance and do not limit the components. These terms are only used to distinguish one component from another. For example, a first user device and a second user device may indicate different user devices from each other, regardless of the order or importance of the devices. A first component may be denoted a second component and vice versa without departing from the scope of this disclosure.

It will be understood that, when an element (such as a first element) is referred to as being (operatively or communicatively) “coupled with/to” or “connected with/to” another element (such as a second element), it can be coupled or connected with/to the other element directly or via a third element. In contrast, it will be understood that, when an element (such as a first element) is referred to as being “directly coupled with/to” or “directly connected with/to” another element (such as a second element), no other element (such as a third element) intervenes between the element and the other element.

As used here, the phrase “configured (or set) to” may be interchangeably used with the phrases “suitable for,” “having the capacity to,” “designed to,” “adapted to,” “made to,” or “capable of” depending on the circumstances. The phrase “configured (or set) to” does not essentially mean “specifically designed in hardware to.” Rather, the phrase “configured to” may mean that a device can perform an operation together with another device or parts. For example, the phrase “processor configured (or set) to perform A, B, and C” may mean a generic-purpose processor (such as a CPU or application processor) that may perform the operations by executing one or more software programs stored in a memory device or a dedicated processor (such as an embedded processor) for performing the operations.

The terms and phrases as used here are provided merely to describe some embodiments of this disclosure but not to limit the scope of other embodiments of this disclosure. It is to be understood that the singular forms “a,” “an,” and “the” include plural references unless the context clearly dictates otherwise. All terms and phrases, including technical and scientific terms and phrases, used here have the same meanings as commonly understood by one of ordinary skill in the art to which the embodiments of this disclosure belong. It will be further understood that terms and phrases, such as those defined in commonly-used dictionaries, should be interpreted as having a meaning that is consistent with their meaning in the context of the relevant art and will not be interpreted in an idealized or overly formal sense unless expressly so defined here. In some cases, the terms and phrases defined here may be interpreted to exclude embodiments of this disclosure.

Definitions for other certain words and phrases may be provided throughout this patent document. Those of ordinary skill in the art should understand that in many if not most instances, such definitions apply to prior as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 illustrates an example network configuration including an electronic device according to this disclosure;

FIG. 2 illustrates an example electronic device in accordance with an embodiment of this disclosure;

FIG. 3 illustrates an example architecture of an LLM-based health monitoring system in accordance with an embodiment of this disclosure;

FIG. 4 illustrates an example pipeline of an LLM-based health monitoring system in accordance with an embodiment of this disclosure;

FIG. 5 illustrates an example architecture of an LLM agent in accordance with an embodiment of this disclosure;

FIG. 6 illustrates an example architecture of an LLL intent agent of an LLM-based health monitoring system in accordance with an embodiment of this disclosure;

FIG. 7 illustrates an example architecture of an LLM health agent of an LLM-based health monitoring system in accordance with an embodiment of this disclosure;

FIG. 8 illustrates an example architecture of an LLM insight agent of an LLM-based health monitoring system in accordance with an embodiment of this disclosure;

FIG. 9 illustrates an example user-specific health information output by an LLM-based health monitoring system in accordance with an embodiment of this disclosure; and

FIG. 10 illustrates an example flow chart for an LLM-based health monitoring method in accordance with an embodiment of this disclosure.

### DETAILED DESCRIPTION

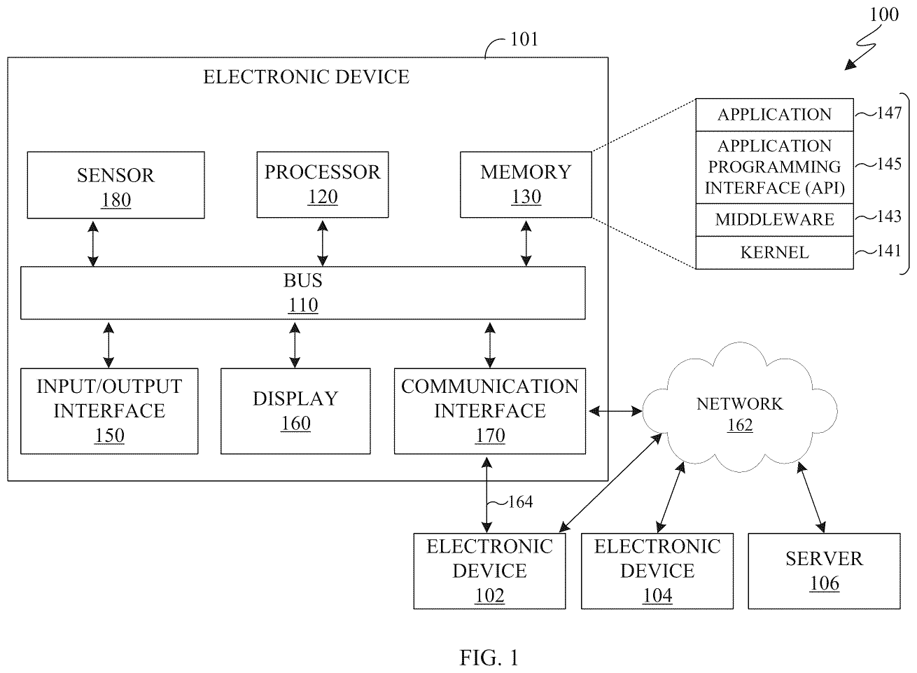

FIGS. 1 through 10, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably-arranged wireless communication system or device.

As the health monitoring technologies continue to evolve to meet the needs of modern healthcare, they have encountered certain problems and limitations. For example, some health monitoring systems may lack advanced analytics, leading to difficulties in identifying patterns and predicting health risks of the users. Some health monitoring systems may encounter delayed response to health issues since relevant data may not be readily available or analyzed in real-time. Some monitoring systems can be inaccessible to rural or underserved populations due to cost, location or lack of healthcare providers. Some health monitoring systems may create data silos, leading to significant challenges in integrating data from multiple sources. Some health monitoring systems may not enable patients to take an active role in their healthcare.

Various embodiments in this disclosure may resolve these problems and limitations by providing an LLM-based health monitoring system. Large Language Models (LLMs) may be utilized to resolve some of these problems and limitations. The LLMs can perform complex data analysis, identifying patterns and predicting health risks more accurately. The LLMs can help personalize treatment plans by analyzing individual patient data, medical history, and lifestyle factors. The LLMs can integrate data from diverse sources, breaking down silos and providing a comprehensive view of patient health. The LLMs can learn from data and improve their performance over time, ensuring that health monitoring systems stay up-to-date and effective. The LLMs can facilitate remote monitoring, expanding access to healthcare services, especially for rural or underserved populations.

The LLM-based health monitoring system in accordance with the present disclosure may provide continuous, real-time monitoring by utilizing multiple LLM agents operating in a sequential multi-agent structure. The system may continuously process data from wearable devices and sensors to create personal health data, utilize this health data and the multiple LLMs to analyze the data and generate personal health insights for the user and healthcare providers in real-time. The multiple LLM agents may learn from the user data and improve their performance over time, ensuring that the health monitoring system remains up-to-date, personalized and effective. By providing integrated data sources, interactive monitoring sessions, and personalized health insights in real-time, the health monitoring system may provide an efficient, accurate, and user-friendly health monitoring solution as described in detail below.

FIG. 1 illustrates an example network configuration 100 including an electronic device according to this disclosure. The embodiment of the network configuration 100 shown in FIG. 1 is for illustration only. Other embodiments of the network configuration 100 could be used without departing from the scope of this disclosure.

According to embodiments of this disclosure, an electronic device 101 may be included in the network configuration 100. The electronic device 101 can include at least one of a bus 110, a processor 120, a memory 130, an input/output (I/O) interface 150, a display 160, a communication interface 170, or a sensor 180. In some embodiments, the electronic device 101 may exclude at least one of these components or may add at least one other component. The bus 110 may include a circuit for connecting the components 120-180 with one another and for transferring communications (such as control messages and/or data) between the components.

The processor 120 may include one or more of a central processing unit (CPU), an application processor (AP), or a communication processor (CP). The processor 120 may be able to perform control on at least one of the other components of the electronic device 101 and/or perform an operation or data processing relating to communication. In some embodiments, the processor 120 can be a graphics processor unit (GPU), or a neural processing unit (NPU). As described in more detail below, the processor 120 may perform one or more operations to support LLM-based health monitoring in wireless network systems.

The memory 130 can include a volatile and/or non-volatile memory. For example, the memory 130 can store commands or data related to at least one other component of the electronic device 101. According to embodiments of this disclosure, the memory 130 can store software and/or a program 140. The program 140 may include, for example, a kernel 141, middleware 143, an application programming interface (API) 145, and/or an application program (or “application”) 147. At least a portion of the kernel 141, middleware 143, or API 145 may be denoted an operating system (OS).

The kernel 141 can control or manage system resources (such as the bus 110, processor 120, or memory 130) used to perform operations or functions implemented in other programs (such as the middleware 143, API 145, or application 147). The kernel 141 may provide an interface that allows the middleware 143, the API 145, or the application 147 to access the individual components of the electronic device 101 to control or manage the system resources. The application 147 may support one or more functions for LLM based health monitoring in wireless network systems as discussed below. These functions can be performed by a single application or by multiple applications that each carry out one or more of these functions. In some embodiments, the application 147 may include LLM agents configured to generate user-specific health information based on user data. The middleware 143 can function as a relay to allow the API 145 or the application 147 to communicate data with the kernel 141, for instance. A plurality of applications 147 can be provided. The middleware 143 may be able to control work requests received from the applications 147, such as by allocating the priority of using the system resources of the electronic device 101 (like the bus 110, the processor 120, or the memory 130) to at least one of the plurality of applications 147. The API 145 may be an interface allowing the application 147 to control functions provided from the kernel 141 or the middleware 143. For example, the API 145 may include at least one interface or function (such as a command) for filing control, window control, image processing, or text control.

The I/O interface 150 may serve as an interface that can, for example, transfer commands or data input from a user or other external devices to other component(s) of the electronic device 101. The I/O interface 150 can also output commands or data received from other component(s) of the electronic device 101 to the user or the other external device.

The display 160 may include, for example, a liquid crystal display (LCD), a light emitting diode (LED) display, an organic light emitting diode (OLED) display, a quantum-dot light emitting diode (QLED) display, a microelectromechanical systems (MEMS) display, or an electronic paper display. The display 160 can also be a depth-aware display, such as a multi-focal display. The display 160 may be able to display, for example, various contents (such as text, images, videos, icons, or symbols) to the user. The display 160 can include a touchscreen and may receive, for example, a touch, gesture, proximity, or hovering input using an electronic pen or a body portion of the user.

The communication interface 170, for example, may be able to set up communication between the electronic device 101 and an external electronic device (such as a first external electronic device 102, a second external electronic device 104, or a server 106). For example, the communication interface 170 can be connected with a network 162 or 164 through wireless or wired communication to communicate with the external electronic device. The communication interface 170 can be a wired or wireless transceiver or any other component for transmitting and receiving signals. The communication interface 170 can also include a radar transceiver that is configured to transmit and receive signals for detecting and ranging purposes such as millimeter wave (mmWave) signals.

The wireless communication may be able to use at least one of, for example, long term evolution (LTE), long term evolution-advanced (LTE-A), 5th generation wireless system (5G), millimeter-wave or 60 GHz wireless communication, Wireless USB, code division multiple access (CDMA), wideband code division multiple access (WCDMA), universal mobile telecommunication system (UMTS), wireless broadband (WiBro), or global system for mobile communication (GSM), as a cellular communication protocol. The wired connection can include, for example, at least one of a universal serial bus (USB), high definition multimedia interface (HDMI), recommended standard 232 (RS-232), or plain old telephone service (POTS). The network 162 or 164 may include at least one communication network, such as a computer network (like a local area network (LAN) or wide area network (WAN)), Internet, or a telephone network.

The electronic device 101 may further include one or more sensors 180 that can meter a physical quantity or detect an activation state of the electronic device 101 and convert metered or detected information into an electrical signal. For example, one or more sensors 180 can include one or more cameras or other imaging sensors for capturing images of scenes. The sensor(s) 180 can also include one or more buttons for touch input, a gesture sensor, a gyroscope or gyro sensor, an air pressure sensor, a magnetic sensor or magnetometer, an acceleration sensor or accelerometer, a grip sensor, a proximity sensor, a color sensor (such as a red green blue (RGB) sensor), a bio-physical sensor, a temperature sensor, a humidity sensor, an illumination sensor, an ultraviolet (UV) sensor, an electromyography (EMG) sensor, an electroencephalogram (EEG) sensor, an electrocardiogram (ECG) sensor, an infrared (IR) sensor, an ultrasound sensor, an iris sensor, or a fingerprint sensor. The sensor(s) 180 can further include an inertial measurement unit, which can include one or more accelerometers, gyroscopes, and other components. In addition, the sensor(s) 180 can include a control circuit for controlling at least one of the sensors included here. Any of these sensor(s) 180 can be located within the electronic device 101.

In some embodiments, the electronic device 101 can be a wearable device or an electronic device-mountable wearable device (such as an HMD). For example, the electronic device 101 may represent an XR wearable device, such as a headset or smart eyeglasses. In other embodiments, the first external electronic device 102 or the second external electronic device 104 can be a wearable device or an electronic device-mountable wearable device (such as an HMD). In those other embodiments, when the electronic device 101 is mounted in the electronic device 102 (such as the HMD), the electronic device 101 can communicate with the electronic device 102 through the communication interface 170. The electronic device 101 can be directly connected with the electronic device 102 to communicate with the electronic device 102 without involving with a separate network.

The first external electronic device 102 can be a device of the same or a different type from the electronic device 101. It may include connected health devices (CHDs) that collect, process, and transmit health-related data to the electronic device 101 or the server 106. The CHDs may include wearables (e.g., smart watches and fitness trackers), medical monitors (e.g., continuous glucose monitors, blood pressure monitors and pulse oximeters), medical implants (e.g., pacemakers, defibrillators, and neurostimulators), home health devices (e.g., smart thermometers and sleep monitors) or remote patient monitoring devices (e.g., smart inhalers, cardiac monitors, or weight scales). The CHDs may detect biological signals (e.g., heart rate, temperature, motion), perform data processing of the detected signals and transmit the signals to the electronic device 101 or the server 106 for further processing and analytics. The CHDs may be connected to the electronic device 101 via, e.g., Bluetooth, Wi-Fi, cellular or Zigbee for device-to-device communication.

The second external electronic devices 104 and the server 106 each can be a device of the same or a different type from the electronic device 101. According to certain embodiments of this disclosure, the server 106 may include a group of one or more servers. The server 106 may be a standalone database configured to receive, process and store user data collected from, e.g., the CHDs and the LLM agents. In some embodiments, the server 106 may be a cloud server configured to perform one or more functions of the LLM-based health monitoring. In some embodiments, the server 106 may include one or more LLM agents configured to generate user-specific health information based on user data. Also, according to certain embodiments of this disclosure, all or some of the operations executed on the electronic device 101 can be executed on another or multiple other electronic devices (such as the electronic devices 102 and 104 or server 106). Further, according to certain embodiments of this disclosure, when the electronic device 101 should perform some function or service automatically or at a request, the electronic device 101, instead of executing the function or service on its own or additionally, can request another device (such as electronic devices 102 and 104 or server 106) to perform at least some functions associated therewith. The other electronic device (such as electronic devices 102 and 104 or server 106) may be able to execute the requested functions or additional functions and transfer a result of the execution to the electronic device 101. The electronic device 101 can provide a requested function or service by processing the received result as it is or additionally. To that end, a cloud computing, distributed computing, or client-server computing technique may be used, for example. While FIG. 1 shows that the electronic device 101 includes the communication interface 170 to communicate with the external electronic device 104 or server 106 via the network 162 or 164, the electronic device 101 may be independently operated without a separate communication function according to some embodiments of this disclosure.

The server 106 can include the same or similar components 110-180 as the electronic device 101 (or a suitable subset thereof). The server 106 can support to drive the electronic device 101 by performing at least one of operations (or functions) implemented on the electronic device 101. For example, the server 106 can include a processing module or processor that may support the processor 120 implemented in the electronic device 101. As described in more detail below, the server 106 may perform one or more operations to support LLM-based health monitoring in wireless network systems.

Although FIG. 1 illustrates one example of a network configuration 100 including an electronic device 101, various changes may be made to FIG. 1. For example, the network configuration 100 could include any number of each component in any suitable arrangement. In general, computing and communication systems come in a wide variety of configurations, and FIG. 1 does not limit the scope of this disclosure to any particular configuration. Also, while FIG. 1 illustrates one operational environment in which various features disclosed in this patent document can be used, these features could be used in any other suitable system.

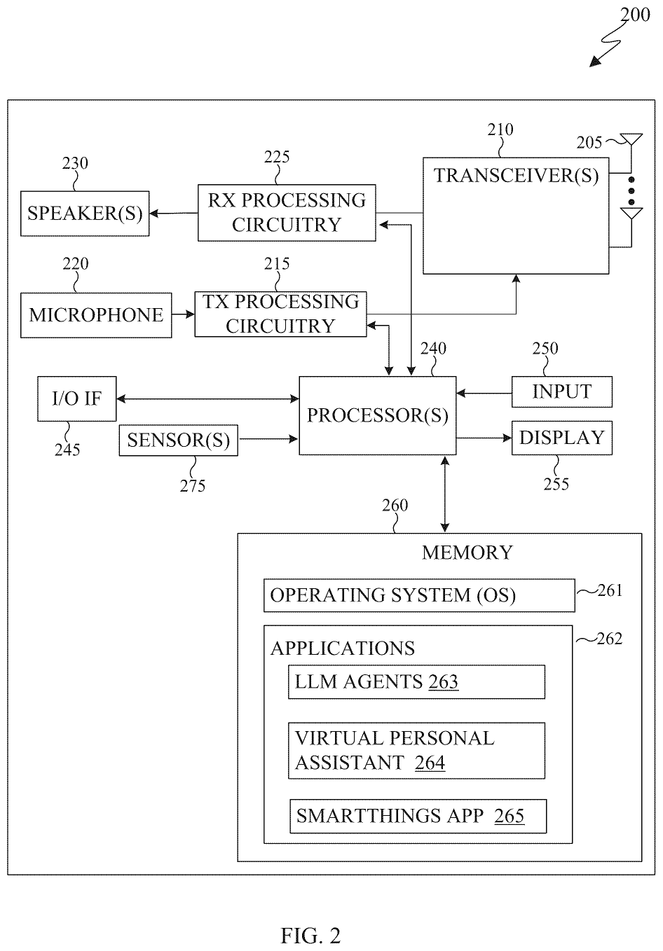

FIG. 2 illustrates an example electronic device in accordance with an embodiment of this disclosure. In particular, FIG. 2 illustrates an example electronic device 200, and the electronic device 200 could represent one or more of the external electronic devices 102-104 or the server 106 in FIG. 1. The electronic device 200 can be a mobile communication device, such as, for example, a mobile station, a subscriber station, a wireless terminal, a desktop computer, a portable electronic device (similar to a mobile device 108, a PDA, a laptop computer, or a tablet computer), a wearable device or an electronic device-mountable wearable device (such as an HMD 730 shown in FIG. 7), a robot, and the like.

As shown in FIG. 2, the electronic device 200 may include transceiver(s) 210, transmit (TX) processing circuitry 215, a microphone 220, and receive (RX) processing circuitry 225. The transceiver(s) 210 can include, for example, a RF transceiver, a BLUETOOTH transceiver, a WiFi transceiver, a ZIGBEE transceiver, an infrared transceiver, and various other wireless communication signals. The electronic device 200 may also include a speaker 230, a processor 240, an input/output (I/O) interface (IF) 245, an input 250, a display 255, a memory 260, and a sensor 275. The memory 260 may include an operating system (OS) 261, and one or more applications 262.

The transceiver(s) 210 can include an antenna array 205 including numerous antennas. The transceiver(s) 210 can include or can be the same as or similar to the radar transceiver configured to receive and transmit mmWave signals. The antennas of the antenna array can include a radiating element composed of a conductive material or a conductive pattern formed in or on a substrate. The transceiver(s) 210 may transmit and receive a signal or power to or from the electronic device 200. The transceiver(s) 210 may receive an incoming signal transmitted from an access point (such as a base station, WiFi router, or BLUETOOTH device) or other device of the network configuration 100 (such as a WiFi, BLUETOOTH, cellular, 5G, 6G, LTE, LTE-A, WiMAX, or any other type of wireless network). The transceiver(s) 210 may down-convert the incoming RF signal to generate an intermediate frequency or baseband signal. The intermediate frequency or baseband signal may be sent to the RX processing circuitry 225 that may generate a processed baseband signal by filtering, decoding, and/or digitizing the baseband or intermediate frequency signal. The RX processing circuitry 225 may transmit the processed baseband signal to the speaker 230 (such as for voice data) or to the processor 240 for further processing (such as for web browsing data).

The TX processing circuitry 215 may receive analog or digital voice data from the microphone 220 or other outgoing baseband data from the processor 240. The outgoing baseband data can include web data, e-mail, or interactive video game data. The TX processing circuitry 215 encodes, multiplexes, and/or digitizes the outgoing baseband data to generate a processed baseband or intermediate frequency signal. The transceiver(s) 210 may receive the outgoing processed baseband or intermediate frequency signal from the TX processing circuitry 215 and may up-convert the baseband or intermediate frequency signal to a signal that is transmitted.

The processor 240 can include one or more processors or other processing devices. The processor 240 can execute instructions that are stored in the memory 260, such as the OS 261 in order to control the overall operation of the electronic device 200. For example, the processor 240 could control the reception of downlink (DL) channel signals and the transmission of uplink (UL) channel signals by the transceiver(s) 210, the RX processing circuitry 225, and the TX processing circuitry 215 in accordance with well-known principles. The processor 240 can include any suitable number(s) and type(s) of processors or other devices in any suitable arrangement. For example, in certain embodiments, the processor 240 may include at least one microprocessor or microcontroller. Example types of processor 240 may include microprocessors, microcontrollers, digital signal processors, field programmable gate arrays, application specific integrated circuits, and discrete circuitry. In certain embodiments, the processor 240 can include a neural network.

The processor 240 may be also capable of executing other processes and programs resident in the memory 260, such as operations that receive and store data. The processor 240 can move data into or out of the memory 260 as required by an executing process. In certain embodiments, the processor 240 may be configured to execute the one or more applications 262 based on the OS 261 or in response to signals received from external source(s) or an operator. Example, applications 262 can include one or more LLM agents 263, a virtual personal assistant 264 (such as a chatbot or an AI assistant), a SmartThings application 265, a multimedia player (such as a music player or a video player), a phone calling application, a video conferencing application, a text messaging application, and the like.

The processor 240 may be also coupled to the I/O interface 245 that provides the electronic device 200 with the ability to connect to other devices, such as client devices 106-114. The I/O interface 245 may be the communication path between these accessories and the processor 240.

The processor 240 may be also coupled to the input 250 and the display 255. The operator of the electronic device 200 can use the input 250 to enter data or inputs into the electronic device 200. The input 250 can be a keyboard, touchscreen, mouse, track ball, voice input, or other device capable of acting as a user interface to allow a user in interact with the electronic device 200. For example, the input 250 can include voice recognition processing, thereby allowing a user to input a voice command. In another example, the input 250 can include a touch panel, a (digital) pen sensor, a key, or an ultrasonic input device. The touch panel can recognize, for example, a touch input in at least one scheme, such as a capacitive scheme, a pressure sensitive scheme, an infrared scheme, or an ultrasonic scheme. The input 250 can be associated with the sensor(s) 265, a camera, and the like, which provide additional inputs to the processor 240. The input 250 can also include a control circuit. In the capacitive scheme, the input 250 can recognize touch or proximity.

The display 255 can be a liquid crystal display (LCD), light-emitting diode (LED) display, organic LED (OLED), active-matrix OLED (AMOLED), or other display capable of rendering text and/or graphics, such as from websites, videos, games, images, and the like. The display 255 can be a singular display screen or multiple display screens capable of creating a stereoscopic display. In certain embodiments, the display 255 can be a heads-up display (HUD).

The memory 260 may be coupled to the processor 240. Part of the memory 260 could include a RAM, and another part of the memory 260 could include a Flash memory or other ROM. The memory 260 can include persistent storage (not shown) that represents any structure(s) capable of storing and facilitating retrieval of information (such as data, program code, and/or other suitable information). The memory 260 can contain one or more components or devices supporting longer-term storage of data, such as a read only memory, hard drive, Flash memory, or optical disc.

The electronic device 200 may further include one or more sensors 275 that can meter a physical quantity or detect an activation state of the electronic device 200 and convert metered or detected information into an electrical signal. For example, the sensor 275 can include one or more buttons for touch input, a camera, a gesture sensor, optical sensors, cameras, one or more inertial measurement units (IMUs), such as a gyroscope or gyro sensor, and an accelerometer. The sensor 275 can also include an air pressure sensor, a magnetic sensor or magnetometer, a grip sensor, a proximity sensor, an ambient light sensor, a bio-physical sensor, a temperature/humidity sensor, an illumination sensor, an Ultraviolet (UV) sensor, an Electromyography (EMG) sensor, an Electroencephalogram (EEG) sensor, an Electrocardiogram (ECG) sensor, an IR sensor, an ultrasound sensor, an iris sensor, a fingerprint sensor, a color sensor (such as a Red Green Blue (RGB) sensor), a Wi-Fi sensing device and the like. The sensor 275 can further include control circuits for controlling any of the sensors included therein. Any of these sensor(s) 275 may be located within the electronic device 200 or within a secondary device operably connected to the electronic device 200.

Although FIG. 2 illustrates one example of electronic device 200, various changes can be made to FIG. 2. For example, various components in FIG. 2 can be combined, further subdivided, or omitted and additional components can be added according to particular needs. As a particular example, the processor 240 can be divided into multiple processors, such as one or more central processing units (CPUs), one or more graphics processing units (GPUs), one or more neural networks, and the like. Also, while FIG. 2 illustrates the electronic device 200 configured as a mobile telephone, tablet, or smartphone, the electronic device 200 can be configured to operate as other types of mobile or stationary devices.

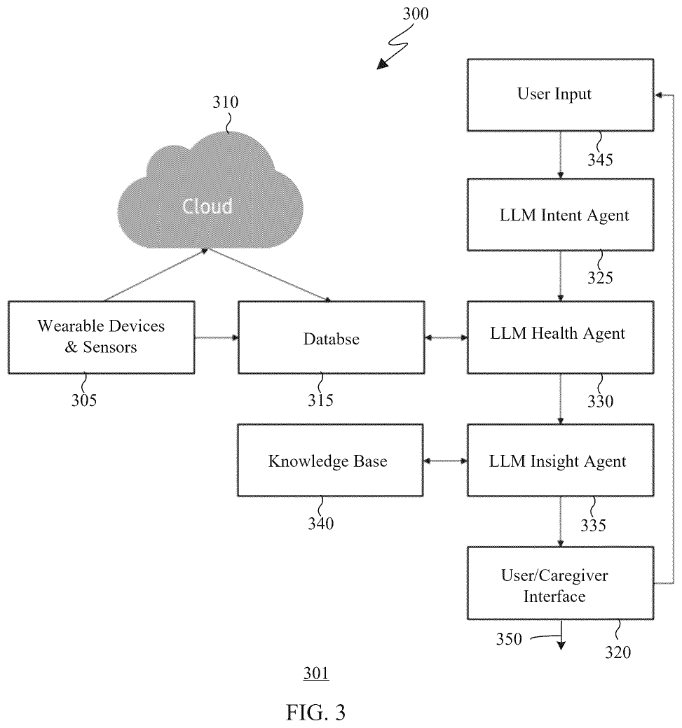

FIG. 3 illustrates an example architecture 300 of an LLM-based health monitoring system 301 in accordance with example embodiments of the present disclosure. The example system 301 shown in FIG. 3 is provided for illustrative purposes only, and thus can change as appropriate without departing from the scope of the present disclosure. For example, one or more components and/or devices of the system 301 can be removed or additional components and/or devices may be added to the system 301.

The system 301 as illustrated in FIG. 3 may include wearable devices and sensors 305, a cloud server 310, a database 315, a user interface 320, multiple LLM agents, and a knowledge base 340. The multiple agents may include an LLM intent agent 325, an LLM health agent 330 and an LLM insight agent 335.

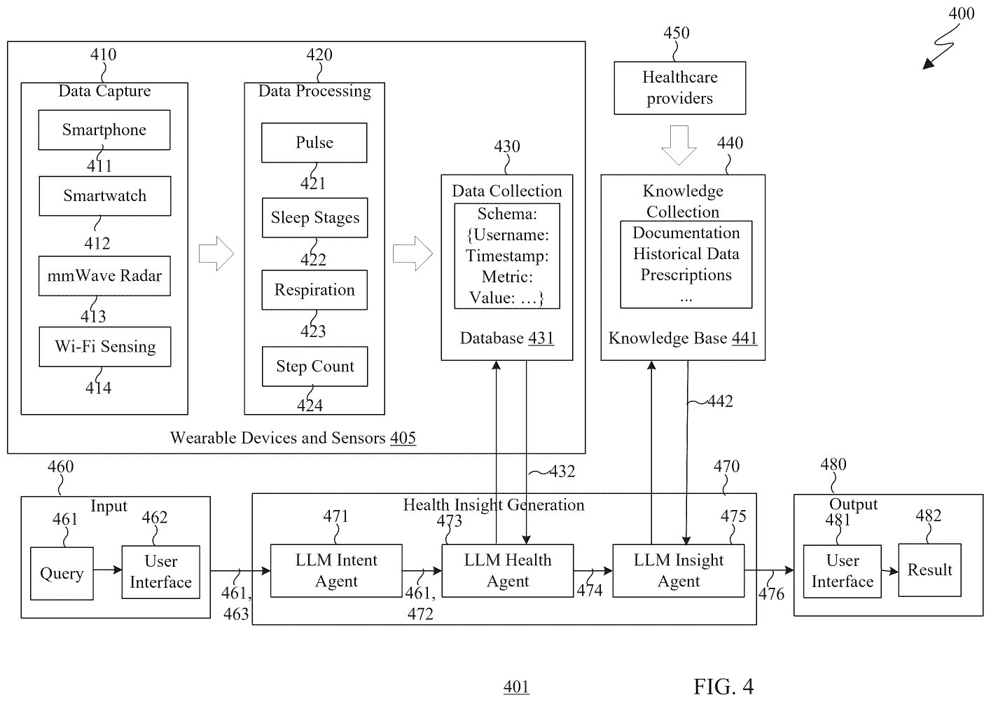

The wearable devices and sensors 305 may be the first external electronic device 102 and/or the sensors 180 of the electronic device 101 of FIG. 1. The wearable devices may be a category of hardware and software that can generate vital signs of a human subject. The sources of sensing signals may not be limited to the wearable devices, but also can include sensors that monitor a human subject. The wearable devices and sensors 305 may capture signals including photoplethysmography (PSG), electrocardiogram (ECG), radar, GPS, IMU, and audio. The raw signals may lack interpretability and require further signal processing as discussed further in FIG. 4.

The cloud server 310 may provide a service that stores and processes IoT data remotely with more powerful computing resources and databases as compared to the wearable devices and sensors 305. While the cloud 310 may be optional, it can be utilized for remote tasks and tasks that require high performance computing or data storage.

The database 315 may collect and store user data and can be accessed by an LLM agent (the LLM health agent 330). The database 315 can be included in the wearable devices and sensors 305 or the cloud server 310. The user data can thus either be directly streamed from the wearable devices and sensors 305 or synchronized from the cloud server 310.

The user interface 320 may be, e.g., the input/output interface 150 or 245 of the electronic device 101 or 200 of FIGS. 1 and 2. The user interface 320 may be an interactive interface between human users and the health monitoring system 301 and utilized to input a user query 345 or output a response 350 to the user query 345. It can include multiple forms such as a chatbot, dashboard, and spreadsheet. The user query 350 may include a user input from the user to the LLM intent agent 325. Example user queries 345 may be “Did I sleep well last night?”, “How can I improve my body exercise?”, “Has the risk of heart attack increased for patient X?”.

The multiple LLM agents may be, e.g., the LLM agents 263 of the electronic device 200 of FIG. 2. An LLM intent agent 325 may read the user query 345, determine a user intent based on the user query 345, and forward the user intent to the LLM health agent 330. The function of this agent 325 may be to generate a user intent from the user query 345 in natural language. For example, based on a user query of “Did I sleep well last night,” it may generate a user intent as follows: {“user”: “user”, “time”: “last_night”, “query”: “sleep”, “input”: “Did I sleep well last night?”}. In another example, based on a user query of “Has the risk of heart attack increased for patient X?”, it may generate a user intent as follows: {“user”: “X”, “time”: “now”, “query”: “heart_attack_risk”, “input”: “Has the risk of heart attack increased for patient X?”}.

The LLM health agent 330 may access the relevant user data based on the user intent received from the LLM intent agent 325, and generate analytical results based on the relevant user data. This agent 330 may need to use tools and/or functions such as a code interpreter to analyze the data. It is noted that multiple LLM health agents may co-exist in the system 301, each LLM health agent focusing on different user data types (such as a heart rate, a gait, and sleep stages).

An LLM insight agent 335 may generate an overall answer 350 to the user query 345. It may gather information from both the analytical results received from LLM health agent 330 and user-specific healthcare data from a knowledge base 340. It may also update the knowledge base 340 as needed.

The knowledge base 340 may be a database including user-specific healthcare knowledge, historical data, medical history, documentations, etc. This may provide an essential part of personalization of the system 301. Authorized healthcare providers can update the knowledge base 340 if necessary.

Thus, the multiple LLM agents may operate in a sequential multi-agent structure with the LLM intent agent 325 configured to understand the user's need, the LLM health agent 330 configured to analyze the relevant user data (relevant user data types) and the LLM insight agent 335 configured to generate a health insight based on the analytical results received from the LLM health agent 330 and the user-specific healthcare data retrieved from the knowledge base 340. Each component of the system 301 and corresponding operation is illustrated further in detail with reference to FIG. 4.

FIG. 4 illustrates an example pipeline 400 for an LLM-based health monitoring system 401 in accordance with an embodiment of this disclosure. The pipeline 400 shown in FIG. 4 may be performed using the electronic device 101 or 102 of FIG. 1 or any other suitable device(s). It will be understood that the example pipeline 400 as shown in FIG. 4 is provided for illustrative purposes only, and thus can vary as appropriate without departing from the scope of the present disclosure. It will be also understood that the components and/or devices of the health monitoring system 401 as illustrated in FIG. 4 may vary. For example, one or more components and/or devices of the system 401 can be removed or additional components and/or devices may be added to the system 401.

As shown in FIG. 4, the pipeline 400 may include a data capture operation 410, a data processing operation 420, a data collection operation 430, a knowledge collection operation 440, an input operation 460, a health insight generation operation 470, and an output operation 480. The data capture operation 410 may generally operate to capture user data (e.g., different user data types) by the wearable devices and sensors 405. The wearable devices and sensors 405 may include a smartphone 411, a smartwatch 412, a mmWave Radar 413, a Wi-Fi sensing device 414 and other appropriate CHDs. The smartphone 411 may be a device of the same or a different type from the electronic device 101 and include user activity monitoring applications (e.g., sleep stage tracker). The smartwatch 412 may be worn by a user and include, e.g., a heart rate monitor that continuously tracks the user's heart rate. The mmWave radar 413 may be used for non-intrusive monitoring and detect user activity including a fall. The Wi-Fi sensing device 414 may utilize Wi-Fi based sensing (e.g., using channel state information) to detect, e.g., a motion, a respiratory rate, a heart rate, etc.

The wearable devices and sensors 405 may continuously capture signals and perform processing on the signals to extract user data. Since the raw signals may lack interpretability, they may undergo data processing operation 420.

The data processing operation 420 may generally operate to process the captured user data. This may include the wearable devices and sensors 405 performing signal processing on the raw signals to transform them into interpretable data (e.g., cleaned waveforms). The wearable devices and sensors 405 may further process the signals to derive context information such as vital signs or other user data types. The context information may include, e.g., pulse 421, sleep stages 422, respiration 423, and step counts 424.

The data collection operation 430 may generally operate to continuously collect and store the processed user data (the context information) in a database 431. This may include the wearable devices and sensors 405 storing the collected user data in a local database. Optionally, the data collection operation 420 may include a remote server (e.g., a cloud server 310 of FIG. 3) collecting, processing and storing the captured user data. The remote server may be communicatively coupled to the wearable devices and sensors 405 and configured to process, synchronize and store the user data collected by the wearable devices and sensors 405. Thus, the collected user data can be fully accessed by the health monitoring system 401 via direct streaming from the wearable devices & sensors 405 through or synchronization from the cloud server. The database 431 (local or remote) may include a database with schemas. That is, the collected user data may have a structured framework (a schema) that can be used by the LLM health agent 473. An example schema may be {Username: Timestamp: Metric: Value: . . . }. The schema may enable the LLM health agent 473) to identify, process, and analyze relevant user data.

The knowledge collection operation 440 may generally operate to process, store and retrieve user-specific healthcare data and include a knowledge base 441. The knowledge base 441 may be a specialized database that is a structured, dynamic, and secure repository of user-specific healthcare data for one or more users. The knowledge base 441 can be fully accessed by the LLM insight agent 475 and an authorized healthcare provider(s) 450. The user-specific healthcare data may include one or more users' historical data, medical history, prescriptions, lab results, device data, behavioral and psychometric data and associated documentations such as medical literature, device specifications, medical reports, and healthcare provider's notes. The knowledge base 441 may be a vector database configured to enable the LLM insight agent 475 to efficiently retrieve semantically similar data. The authorized healthcare provider (such as physicians, nurses and caregivers) 450 can share and/or update the knowledge base 441 as needed.

The input operation 460 may generally operate to receive a user input, and include a user interface 462. The user input may include a user query 461 such as “Did I sleep well last night?”, “How can I improve my body exercise?”, and “Has the risk of heart attack increased for patient X?”, entered by a user or a healthcare provider 450 via the user interface 462. Upon entry of a user query 461 by a user, the user interface 462 may wrap the user query 461 with a prompt 463.

The health insight generation operation 470 may operate to generate a user-specific health information and include multiple LLM agents. The multiple LLM agents may include the LLM intent agent 471, the LLM health agent 473, and the LLM insight agent 475. Each LLM agent may use a prompt to maintain consistency with demonstrations guiding specific tasks. The operations of each LLM agent are described in detail below.

The LLM intent agent 471 may receive a user query 461 wrapped in the prompt 463 from the user interface 462. The LLM intent agent 471 may then read the user query 461, determine a user intent 472 based on the user query 461, generate the user intent 472 in natural language format, and pass the user intent 472 to the LLM health agent 473.

The LLM health agent 473 may receive information including the user query 461 and the user intent 472 from the LLM intent agent 471. The LLM health agent 473 may access the database 431 to retrieve relevant user data 432 (streamed or synthesized) based on the user intent 472. The LLM health agent 473 may then analyze the relevant user data 432 to generate analytical results 474. The LLM health agent 473 may use tools or functions such as a code interpreter to analyze the relevant user data 432. The LLM health agent 373 may include multiple LLM health agents co-existing and performing analyses of specific user data types.

The LLM insight agent 475 may generate a user-specific health information 476 based on the analytical results 474 and relevant user-specific healthcare data 442. This may include the LLM insight agent 475 receiving the analytical results 474 from the LLM health agent 473, retrieving the relevant user-specific healthcare data 442 from the knowledge base 441, and synthesizing the current monitoring session data to generate the user-specific health information 476. The current monitoring session data may include the user query 461, the user intent 472, the relevant user data 432, the analytical result 474, and the relevant user-specific healthcare data 442. The LLM insight agent 475 may output the user-specific health information 476 in a visual (e.g., texts, graphs, charts, tables, etc.), audio or any other format as appropriate. The user-specific health information may include an overview of the user health condition, risk analysis of diseases, suggestion and coaching on daily life habits. The LLM insight agent 475 may also issue threshold-based alerts in real-time based on the user-specific heath information 476. It may also provide a recommendation or an alert based on the analytical results. It may also cross-check the recommendations or the alerts against regulatory guidelines or recent studies to ensure compliance and accuracy.

The current monitoring session result 482 including the user-specific health information 476 may be stored in the long term memory of the LLM insight agent 475. Further, the LLM insight agent 475 may update the knowledge base 441 with the current monitoring session result 482 or as needed. Note that the current monitoring session data may be stored in the short-term memory of the LLM insight agent 475 during the current monitoring session and then in the long-term memory after the completion of the current monitoring session.

Thus, each LLM agent may focus on a specific task, improving efficiency and maintainability. Further, each agent may be swapped or upgraded without disrupting the entire health monitoring system 401. Additional LLM agents can be added to handle new tasks or data sources (e.g., integrating genomic data for insomnia). The sequential operations by the LLM agents (e.g., in a daisy chain manner) may allow for iterative refinement, thereby reducing errors. Further, the sequential operations may allow the system 401 to maintain context across various tasks, ensuring personalized and coherent insight outputs. Therefore, the LLM-based health monitoring system 401 may allow the LLM agents to collaborate to tailor the health insights and/or alerts based on real-time and historical user data, user-specific healthcare knowledge, and user's lifestyle and/or preferences, resulting in efficient health monitoring, immediate and personalized feedback and maximized user satisfaction.

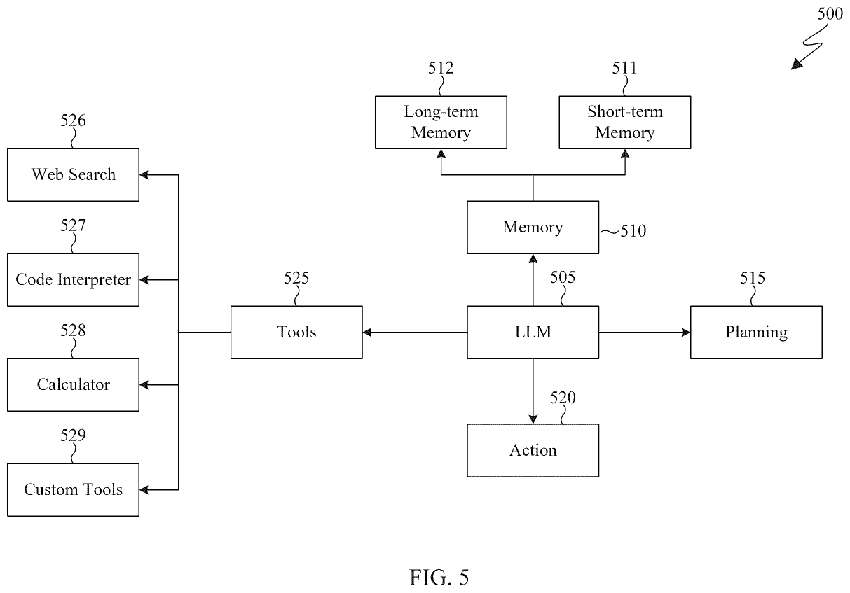

FIG. 5 illustrates an example architecture 500 of an LLM agent of an LLM-based health monitoring system in accordance with an embodiment of this disclosure. The LLM agent as illustrated in FIG. 5 may have an agentic design pattern. The agentic design pattern for LLMs may refer to the design of LLMs as autonomous agents that can interact with their environment, make decisions, and take actions to achieve specific goals. By adopting the agentic design pattern, the LLM agent can become more effective, efficient, and user-friendly. The LLM agent may be built based on off-the-shelf LLMs, and can be an on-device LLM or server-hosted LLM. The example architecture 500 as illustrated in FIG. 5 is one example of the LLM agent and various changes may be made to FIG. 5 as appropriate without departing from the scope of the present disclosure. In the health monitoring system, each LLM agent may be designed on purpose, and include a subset of the components of the architecture 500.

The LLM agent as illustrated in FIG. 5 may include a core LLM 505, memory 510, a planning module 515, an action module 520, and tools 525. The core LLM 505 may be an API from LLM service providers or an open LLM embedded in a framework (e.g., Langchain) to support integration with other components. The memory 510 may store the dialogue history between a user(s) and the LLM agent. The memory 510 may include a short-term memory 511 configured to store current monitoring session information and a long-term memory 512 configured to store previous monitoring session information and/or other relevant historical data. The planning module 515 may establish a plan of actions. That is, with adequate instructions (e.g., Chain-of-Thought) and information from the memory 510, the planning module may assist the LLM agent to break down a task into sub-tasks and complete the sub-tasks sequentially. The action module 520 may provide a response from the LLM agent on a sub-task. The response can be either a text generated based on an input, an immediate input, or calling a tool 525 to complete a sub-task. The tools 525 may provide a means for the LLM agent to interact with the external world and include a web search 526, a code interpreter 527, a calculator 528 and custom tools 529. For example, the LLM agent may plot figures with a code interpreter 527 or query a database with a command.

As used herein, the term “module” may include a unit implemented in hardware, software, or firmware, and may interchangeably be used with other terms, for example, “logic,” “logic block,” “part,” or “circuitry.” A module may be a single integral component, or a minimum unit or part thereof, adapted to perform one or more functions. For example, according to an embodiment, the module may be implemented in a form of an application-specific integrated circuit (ASIC).

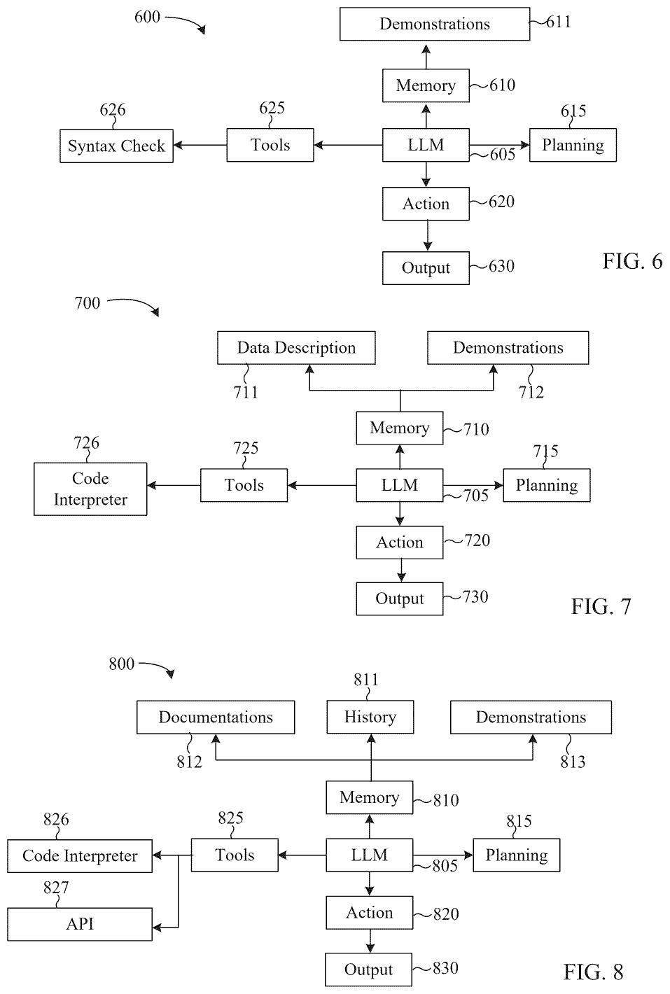

FIG. 6 illustrates an example architecture 600 of an LLL intent agent of an LLM-based health monitoring system in accordance with an embodiment of this disclosure. The example architecture 600 as illustrated in FIG. 6 is one example of the LLM intent agent and various changes may be made to FIG. 6 as appropriate without departing from the scope of the present disclosure.

The example LLM intent agent as illustrated in FIG. 6 may include a core LLM 605, memory 610, a planning module 615, an action module 620, and tools 625 including a syntax check tool 626. The LLM intent agent may learn from demonstrations 611 in the memory (prompt) 610 on how to understand a user intent from an input. The planning module 615 may plan how to parse the user intent from the input. The action module 625 may generate a formatted output (e.g., JSON) 630. The syntax check tool 626 may be utilized to validate the generated output (e.g., text).

The LLM intent agent can be trained to determine a user intent based on an input (a user query) and demonstrations 611 in a prompt. Each demonstration 611 may map a user input to a user intent label. Thus, the LLM intent agent may learn from demonstrations 611 how to understand a user intent from a user input and how to parse the user intent from the input. The demonstrations 611 may be example input-output pairs or scenarios embedded in the prompt, stored in the LLM intent agent's memory. For example, a prompt might include sample queries about insomnia or heart attacks with corresponding user intents and health information to teach the LLM intent agent how to interpret similar user inputs. The LLM intent agent may learn to observe patterns in the demonstrations (e.g., how certain keywords or query structures map to specific user intents) 611 and generalize these patterns to new inputs, improving its ability to classify user intents.

For inferencing, the LLM intent agent may analyze the user query and match it to the closest demonstration in the prompt. It may then identify the user intent as, e.g., “sleep” or “heart_attack_risk.” The demonstrations may be stored in the LLM intent agent's short term memory during the monitoring session. The LLM intent agent's long term memory may include previous dialogues or interactions. The LLM intent agent may incorporate the prior dialogues or interactions as additional demonstrations to refine its understanding of the user intents. Thus, the LLM intent agent may learn to handle diverse inputs without retraining using demonstrations tailored to specific contexts or users, reducing the computational complexity. The demonstrations 611 may be updated to include new user intents. Thus, learning from the demonstrations 611 in memory may enable the LLM intent agent to understand user intents by leveraging example-driven, in-context learning, thereby ensuring precise interpretation of a user query such as “How did I sleep last night?”

FIG. 7 illustrates an example architecture 700 of an LLM health agent of an LLM-based health monitoring system in accordance with an embodiment of this disclosure. The example architecture 700 as illustrated in FIG. 7 is one example of the LLM health agent and various changes may be made to FIG. 7 as appropriate without departing from the scope of the present disclosure.

The example LLM health agent as illustrated in FIG. 7 may include a core LLM 705, memory 710, a planning module 715, an action module 720, and tools 725 including a code interpreter 726. The LLM health agent may learn from the memory 710 about data description 711 and demonstrations 712 on how to process relevant user data. The planning module 715 may plan how to process and analyze the relevant user data using a code interpreter 726. The action module 720 may process and analyze the relevant user data using the code interpreter 726 based on the plan. To complete the analysis of the relevant user data, the action module 720 may perform a number of iterations. The output 730 may include a summary of the analysis results.

The LLM health agent may be trained to identify a user data corresponding to a user intent label, process the corresponding user data, and generate a data analysis result using data descriptions 711 and demonstrations 712 in the memory 710 (a prompt). Each demonstration 712 may map a user intent label and a corresponding data description 711 to a data analysis result. Thus, the LLM health agent may learn from the memory 710 about data description 711 and demonstrations 712. It may access the memory 710 including a short term memory and a long term memory (persistent memory including the previous dialogues or interactions). A data description 711 may include a structured explanation (e.g., a schema) of the user data and help the LLM health agent to understand the user data's structure and relevant user data (e.g., sleep metrics for insomnia monitoring). Demonstrations 712 may include examples within the prompt showing how to process and analyze similar data. For example, a demonstration 712 may show how to calculate an average sleep duration from a dataset. The LLM health agent may utilize a code interpreter 726 to execute codes in a secure environment and perform calculations and statistical analysis. The LLM health agent may then output analytical results 730.

For inferencing, the LLM health agent may analyze the user intent and match it to the closest data description and demonstration in the prompt. It may then access the relevant user data and analyze the relevant user data, e.g., 5.6 hours of awakening during 8 hours of attempted sleep. The data description 711 and demonstrations 712 may be stored in the LLM health agent's short term memory during the monitoring session. The LLM health agent's long term memory may include previous dialogues, interactions and analytical results. The LLM health agent may incorporate the prior dialogues, interactions and analytical results as additional demonstrations to refine its analysis. Thus, the LLM health agent may learn to handle diverse user queries, intents and data types without retraining, thereby reducing the computational complexity.

FIG. 8 illustrates an example architecture 800 of an LLM insight agent of an LLM-based health monitoring system in accordance with an embodiment of this disclosure. The example architecture 800 as illustrated in FIG. 8 is one example of the LLM insight agent and various changes may be made to FIG. 8 as appropriate without departing from the scope of the present disclosure.

The example LLM insight agent as illustrated in FIG. 8 may include a core LLM 805, memory 810, a planning module 815, an action module 820, and tools 825 including a code interpreter 826 and an API 827. The LLM insight agent may combine information from historical data 811, documentations 812 for wellness, and demonstrations 813 stored in the memory 810. The demonstrations 812 may show how to find insights from an input text and generate answers. The tools 825 such as the code interpreter 826 and the API 827 may be utilized to interact with the relevant database and user interfaces. For example, the API calling to the API 827 may be made to update historical data in the knowledge base or the code interpreter 826 may be utilized to visualize the answers (output) 830.

The LLM insight agent may be trained to combine information including historical data 811 and wellness documentations 812 and generate, using demonstrations 813, user-specific health information based on the combined information. Each demonstration 813 may map a user intent label, a user data corresponding to the user intent label and a data analysis result to user-specific health information. Thus, the LLM insight agent may learn to utilize tools such as the code interpreter 826 and an API 827 to formulate answer and to interact with appropriate databases and user interfaces. The output of the LLM insight agent may include visualizations or tables.

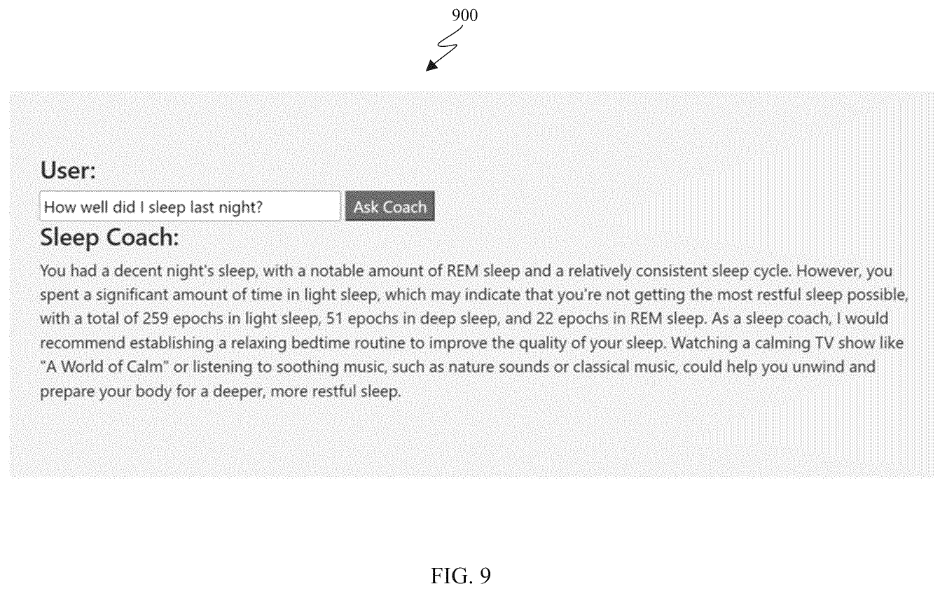

For inferencing, the LLM insight agent may read the analytical results from the LLM health agent and inquire for corresponding entries in the knowledge database. With retrieval augmented generation (RAG), the LLM insight agent may generate strongly personalized texts and/or figures and output the final results 830 on the user interface as shown in FIG. 9.

FIG. 9 illustrates an example user-specific health information 900 output by an LLM insight agent of an LLM-based health monitoring system according to embodiments of the present disclosure. The example information 900 as illustrated in FIG. 9 is for illustrative purposes only, and thus it will be understood that the LLM insight agent may output different user-specific health information based on different user query, user intent, analytical results and/or knowledge data.

The example information 900 may be an example health insight provided in response to a user query of “How well did I sleep last night?”. It may display an output text on a user interface delineating a specific recommendation, e.g., a sleep coaching based on the analytical results from an LLM health agent and user-specific healthcare data from a knowledge base.

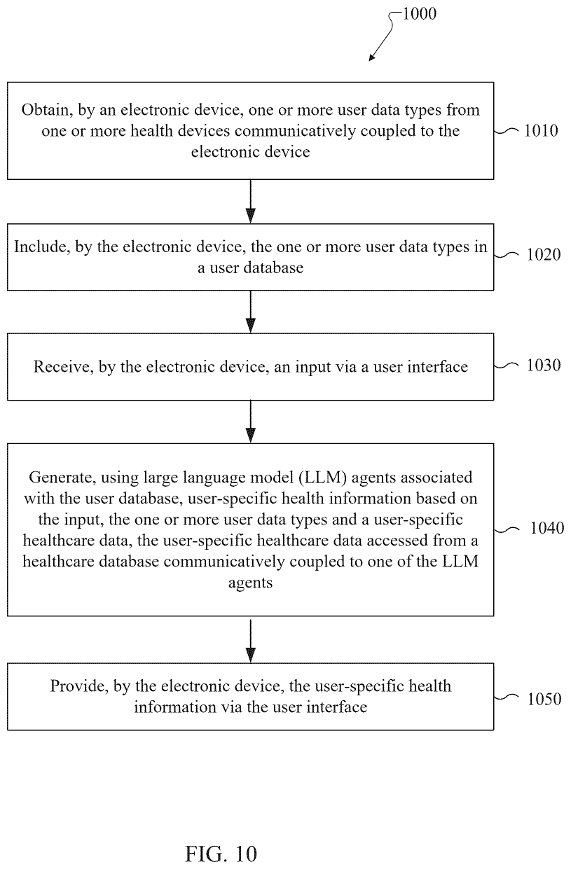

FIG. 10 illustrates an example flow chart for an LLM-based health monitoring method according to embodiments of the present disclosure. An embodiment of the method illustrated in FIG. 10 is for illustration only. One or more of the components illustrated in FIG. 10 may be implemented in specialized circuitry configured to perform the noted functions or one or more of the components may be implemented by one or more processors executing instructions to perform the noted functions. Other embodiments of data preparation could be used without departing from the scope of this disclosure.

As illustrated in FIG. 10, the method 1000 begins at step 1010. At step 1010, an electronic device (e.g., without limitation, an electronic device 101 of FIG. 1) may obtain one or more user data types from one or more health devices (e.g., the wearable devices and sensors) communicatively coupled to the electronic device.

At step 1020, the electronic device may include the one or more user data types in a user database.

At step 1030, the electronic device may receive an input via a user interface.

At step 1040, the electronic device may generate, using large language model (LLM) agents associated with the user database, user-specific health information based on the input, the one or more user data types and a user-specific healthcare data, the user-specific healthcare data accessed from a healthcare database communicatively coupled to one of the LLM agents.

In one embodiment, generating the user-specific health information may further include determining, by a first LLM agent of the LLM agents, a user intent based on the input and a prompt to generate a user intent in a natural language format, accessing, by a second LLM agent of the LLM agents, a corresponding user data from the user database based on the user intent, analyzing, by the second LLM agent, the corresponding user data to generate a data analysis result based on the user intent and the user input, retrieving, by a third LLM agent of the LLM agents, the user-specific healthcare data, synthesizing, by the third LLM agent, a monitoring session data including the user input, the user intent, the corresponding user data, the data analysis result, and the user-specific healthcare data, generating, by the third LLM agent, the user-specific health information based on the synthesized session data, and issuing threshold-based alerts in real-time based on the user-specific health information.

At step 1050, the electronic device may provide the user-specific health information via the user interface.

In one embodiment, the method 1000 may further include training the LLM agents. Training the LLM agents may include training a first LLM agent of the LLM agents to determine a user intent based on a user input and first demonstrations in a first prompt. Each first demonstration may map a user input to a user intent label. Training the LLM agents may also include training a second LLM agent of the LLM agents to identify a user data corresponding to a user intent label, process the corresponding user data, and generate a data analysis result using data descriptions and second demonstrations in a second prompt. Each second demonstration may map a user intent label and a corresponding data description to a data analysis result. Training the LLM agents may include training a third LLM agent of the LLM agents to combine information including historical data and wellness documentations and generate, using third demonstrations, user-specific health information based on the combined information. Each third demonstration may map a user intent label, a user data corresponding to the user intent label and a data analysis result to user-specific health information.

In one embodiment, the method 1000 may further include updating, by at least one LLM agent, the user database with a current monitoring session data and corresponding user-specific health information.

In one embodiment, each LLM agent may have an agentic design pattern including a core LLM, a memory including a dialogue history between a user and the LLM agent, one or more tools including a code interpreter, a calculator, a web search module or a custom tool, a planning module configured to plan a sequence of agent-specific tasks including parsing a user intent from a user input, iteratively analyzing user data corresponding to a user input or finding a user-specific health information based on an input text, and an action module configured to perform a sequence of sub-tasks associated with the agent-specific tasks or trigger the one or more tools to complete the sub-tasks.

In one embodiment, each LLM agent may be an on-device LLM agent or a server-hosted LLM agent.

In one embodiment, the one or more health devices may be communicatively coupled to a server configured to store, process and compute the one or more user data types remotely.

Although the present disclosure has been described with exemplary embodiments, various changes and modifications may be suggested to one skilled in the art. It is intended that the present disclosure encompass such changes and modifications as fall within the scope of the appended claims. None of the description in this application should be read as implying that any particular element, step, or function is an essential element that must be included in the claims scope. The scope of patented subject matter is defined by the claims. None of the description in this application should be read as implying that any particular element, step, or function is an essential element that must be included in the claim scope. The scope of patented subject matter is defined only by the claims.

What is claimed is:

1. A method comprising:
obtaining, by an electronic device, one or more user data types from one or more health devices communicatively coupled to the electronic device;
including, by the electronic device, the one or more user data types in a user database;
receiving, by the electronic device, an input via a user interface;
generating, using large language model (LLM) agents associated with the user database, user-specific health information based on the input, the one or more user data types and a user-specific healthcare data, the user-specific healthcare data accessed from a healthcare database communicatively coupled to one of the LLM agents; and
providing, by the electronic device, the user-specific health information via the user interface.

2. The method of claim 1, wherein generating the user-specific health information comprises:
determining, by a first LLM agent of the LLM agents, a user intent based on the input and a prompt to generate a user intent in a natural language format;
accessing, by a second LLM agent of the LLM agents, a corresponding user data from the user database based on the user intent;
analyzing, by the second LLM agent, the corresponding user data to generate a data analysis result based on the user intent and the user input;
retrieving, by a third LLM agent of the LLM agents, the user-specific healthcare data;
synthesizing, by the third LLM agent, a monitoring session data including the user input, the user intent, the corresponding user data, the data analysis result, and the user-specific healthcare data;
generating, by the third LLM agent, the user-specific health information based on the synthesized session data; and
issuing threshold-based alerts in real-time based on the user-specific health information.

3. The method of claim 1, further comprising training the LLM agents, wherein training the LLM agents comprises:
training a first LLM agent of the LLM agents to determine a user intent based on a user input and first demonstrations in a first prompt, each first demonstration mapping a user input to a user intent label;
training a second LLM agent of the LLM agents to identify a user data corresponding to a user intent label, process the corresponding user data, and generate a data analysis result using data descriptions and second demonstrations in a second prompt, each second demonstration mapping a user intent label and a corresponding data description to a data analysis result; and
training a third LLM agent of the LLM agents to combine information including historical data and wellness documentations and generate, using third demonstrations, user-specific health information based on the combined information, each third demonstration mapping a user intent label, a user data corresponding to the user intent label and a data analysis result to user-specific health information.

4. The method of claim 1, further comprising:
updating, by at least one LLM agent, the user database with a current monitoring session data and corresponding user-specific health information.

5. The method of claim 1, wherein each LLM agent has an agentic design pattern comprising:
a core LLM;
a memory including a dialogue history between a user and the LLM agent;
one or more tools including a code interpreter, a calculator, a web search module or a custom tool;
a planning module configured to plan a sequence of agent-specific tasks including parsing a user intent from a user input, iteratively analyzing user data corresponding to a user input or finding a user-specific health information based on an input text; and
an action module configured to perform a sequence of sub-tasks associated with the agent-specific tasks or trigger the one or more tools to complete the sub-tasks.

6. The method of claim 1, wherein each LLM agent is an on-device LLM agent or a server-hosted LLM agent.

7. The method of claim 1, wherein the one or more health devices are communicatively coupled to a server configured to store, process and compute the one or more user data types remotely.

8. An electronic device comprising:
memory; and
a processor operably coupled to the memory, the processor configured to:
obtain one or more user data types from one or more health devices communicatively coupled to the electronic device;
include the one or more user data types in a user database;
receive an input via a user interface;
generate, using large language model (LLM) agents associated with the user database, user-specific health information based on the input, the one or more user data types and a user-specific healthcare data, the user-specific healthcare data accessed from a healthcare database communicatively coupled to one of the LLM agents; and
provide the user-specific health information via the user interface.

9. The electronic device of claim 8, wherein to generate the user-specific health information, the processor is further configured to:
determine, using a first LLM agent of the LLM agents, a user intent based on the input and a prompt to generate a user intent in a natural language format;
access, using a second LLM agent of the LLM agents, a corresponding user data from the user database based on the user intent;
analyze, using the second LLM agent, the corresponding user data to generate a data analysis result based on the user intent and the user input;
retrieve, using a third LLM agent of the LLM agents, the user-specific healthcare data;
synthesize, using the third LLM agent, a monitoring session data including the user input, the user intent, the corresponding user data, the data analysis result, and the user-specific healthcare data;
generate, using the third LLM agent, the user-specific health information based on the synthesized session data; and
issue threshold-based alerts in real-time based on the user-specific health information.

10. The electronic device of claim 8, wherein:
the processor is configured to train the LLM agents, and
to train the LLM agents, the processor is further configured to:
train a first LLM agent of the LLM agents to determine a user intent based on a user input and first demonstrations in a first prompt, each first demonstration mapping a user input to a user intent label;
train a second LLM agent of the LLM agents to identify a user data corresponding to a user intent label, process the corresponding user data, and generate a data analysis result using data descriptions and second demonstrations in a second prompt, each second demonstration mapping a user intent label and a corresponding data description to a data analysis result; and
train a third LLM agent of the LLM agents to combine information including historical data and wellness documentations and generate, using third demonstrations, user-specific health information based on the combined information, each third demonstration mapping a user intent label, a user data corresponding to the user intent label and a data analysis result to user-specific health information.

11. The electronic device of claim 8, wherein the processor is further configured to update, using at least one LLM agent, the user database with a current monitoring session data and corresponding user-specific health information.

12. The electronic device of claim 8, wherein each LLM agent has an agentic design pattern comprising:
a core LLM;
a memory including a dialogue history between a user and the LLM agent;
one or more tools including a code interpreter, a calculator, a web search module or a custom tool;
a planning module configured to plan a sequence of agent-specific tasks including parsing a user intent from a user input, iteratively analyzing user data corresponding to a user input or finding a user-specific health information based on an input text; and
an action module configured to perform a sequence of sub-tasks associated with the agent-specific tasks or trigger the one or more tools to complete the sub-tasks.

13. The electronic device of claim 8, wherein each LLM agent is an on-device LLM agent or a server-hosted LLM agent.

14. The electronic device of claim 8, wherein the one or more health devices are communicatively coupled to a server configured to store, process and compute the one or more user data types remotely.

15. A non-transitory computer readable medium embodying a computer program, the computer program comprising program code that, when executed by a processor of an electronic device, causes the electronic device to:
obtain one or more user data types from one or more health devices communicatively coupled to the electronic device;
include the one or more user data types in a user database;
receive an input via a user interface;
generate, using large language model (LLM) agents associated with the user database, user-specific health information based on the input, the one or more user data types and a user-specific healthcare data, the user-specific healthcare data accessed from a healthcare database communicatively coupled to one of the LLM agents; and
provide the user-specific health information via the user interface.

16. The non-transitory computer readable medium of claim 15, wherein the program code that, when executed by the processor of the electronic device, causes the electronic device to generate the user-specific health information comprises program code that, when executed by the processor of the electronic device, causes the electronic device to:
determine, using a first LLM agent of the LLM agents, a user intent based on the input and a prompt to generate a user intent in a natural language format;
access, using a second LLM agent of the LLM agents, a corresponding user data from the user database based on the user intent;
analyze, using the second LLM agent, the corresponding user data to generate a data analysis result based on the user intent and the user input;
retrieve, using a third LLM agent of the LLM agents, the user-specific healthcare data;
synthesize, using the third LLM agent, a monitoring session data including the user input, the user intent, the corresponding user data, the data analysis result, and the user-specific healthcare data;
generate, using the third LLM agent, the user-specific health information based on the synthesized session data; and
issue threshold-based alerts in real-time based on the user-specific health information.

17. The non-transitory computer readable medium of claim 15, wherein:
the computer program further comprises program code that, when executed by the processor of the electronic device, causes the electronic device to train the LLM agents, and
the program code that, when executed by the processor of the electronic device, causes the electronic device to train the LLM agents comprises program code that, when executed by the processor of the electronic device, causes the electronic device to:
train a first LLM agent of the LLM agents to determine a user intent based on a user input and first demonstrations in a first prompt, each first demonstration mapping a user input to a user intent label;
train a second LLM agent of the LLM agents to identify a user data corresponding to a user intent label, process the corresponding user data, and generate a data analysis result using data descriptions and second demonstrations in a second prompt, each second demonstration mapping a user intent label and a corresponding data description to a data analysis result; and
train a third LLM agent of the LLM agents to combine information including historical data and wellness documentations and generate, using third demonstrations, user-specific health information based on the combined information, each third demonstration mapping a user intent label, a user data corresponding to the user intent label and a data analysis result to user-specific health information.

18. The non-transitory computer readable medium of claim 15, wherein the computer program further comprises program code that, when executed by the processor of the electronic device, causes the electronic device to update, using at least one LLM agent, the user database with a current monitoring session data and corresponding user-specific health information.

19. The non-transitory computer readable medium of claim 15, wherein each LLM agent has an agentic design pattern comprising:
a core LLM;
a memory including a dialogue history between a user and the LLM agent;
one or more tools including a code interpreter, a calculator, a web search module or a custom tool;
a planning module configured to plan a sequence of agent-specific tasks including parsing a user intent from a user input, iteratively analyzing user data corresponding to a user input or finding a user-specific health information based on an input text; and
an action module configured to perform a sequence of sub-tasks associated with the agent-specific tasks or trigger the one or more tools to complete the sub-tasks.

20. The non-transitory computer readable medium of claim 15, wherein each LLM agent is an on-device LLM agent or a server-hosted LLM agent.
